(19) United States
(12) Patent Application Publication
(10) Pub. No.: US 2025/0296948 A1
(43) Pub. Date: Sep. 25, 2025

(54) SYNTHESIS OF NUCLEOSIDE DERIVATIVES

(71) Applicant: Nucana plc, Edinburgh (GB)

(72) Inventors: Venkata Lakshmi Narasimha Rao Dammalapati, Hyderabad (IN); Suresh Thatipally, Hyderabad (IN); Gordon Kennovin, Edinburgh (GB)

(73) Assignee: Nucana plc, Edinburgh, GB (GB)

(21) Appl. No.: 19/230,886

(22) Filed: Jun. 6, 2025

Related U.S. Application Data
(63) Continuation of application No. PCT/GB2023/051505, filed on Jun. 9, 2023.

(30) Foreign Application Priority Data
Dec. 7, 2022 (IN) 202211070713

(51) Int. Cl. C07H 19/10; A61K 31/7072; C07H 1/02

(52) U.S. Cl. CPC C07H 19/10; A61K 31/7072; C07H 1/02

(57) ABSTRACT

This invention relates to a novel process for the preparation of 5-fluoro-2′-deoxyuridine-5′—O-[1-naphthyl (benzoxy-L-alaninyl)] phosphate (NUC-3373) and derivatives thereof. The NUC-3373 made according to this process is more stable than NUC-3373 made according to alternative processes.

### CROSS-REFERENCE TO RELATED APPLICATIONS

This application is a continuation of International Patent Application No. PCT/GB2023/051505, filed on Jun. 9, 2023; which claims the benefit of priority to Indian Patent Application No. 202211070713, filed Dec. 7, 2022. The entirety of each of these applications is incorporated herein for all purposes.

### FIELD OF THE INVENTION

This invention relates to a novel process for the preparation of 5-fluoro-2′-deoxyuridine-5′-O-[1-naphthyl (benzoxy-L-alaninyl)] phosphate (NUC-3373) and derivatives thereof.

### BACKGROUND

### NUC-3373

Drugs of the ProTide class are masked phosphate derivatives of nucleosides. They have been shown to be particularly potent therapeutic agents in the fields of both antivirals and oncology. Drugs of the ProTide class, more specifically, are prodrugs of monophosphorylated nucleosides. These compounds appear to avoid many of the inherent and acquired resistance mechanisms which limit the utility of the parent nucleosides.

5-fluoro-2′-deoxyuridine-5′-O-[1-naphthyl (benzoxy-L-alaninyl)] phosphate (NUC-3373) and a range of related compounds have shown activity in vitro against a range of cancer models, in many cases and in particular for NUC-3373 that activity was outstanding and far superior to the results obtained with 5-fluorouracil. The addition of the phosphoramidate moiety to the 5-fluorouracil/FUDR molecule confers the specific advantages of delivering the key activated form of the agent (FUDR monophosphate) into the tumour cells. Non-clinical studies have demonstrated that NUC-3373 overcomes the key cancer cell resistance mechanisms associated with 5-FU and its oral pro-drug capecitabine, generating high intracellular levels of the active FUDR monophosphate metabolite, resulting in a much greater inhibition of tumour cell growth. Furthermore, in formal dog toxicology studies, NUC-3373 is significantly better tolerated than 5-FU (see WO 2012/117246; McGuigan et al.; Phosphoramidate ProTides of the anticancer agent FUDR successfully deliver the preformed bioactive monophosphate in cells and confer advantage over the parent nucleoside; J. Med. Chem.; 2011, 54, 7247-7258; and Vande Voorde et al.; The cytostatic activity of NUC-3073, a phosphoramidate prodrug of 5-fluoro-2′-deoxyuridine, is independent of activation by thymidine kinase and insensitive to degradation by phosphorolytic enzymes; Biochem. Pharmacol.; 2011, 82, 441-452).

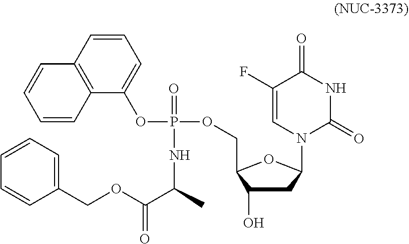

NUC-3373 is typically prepared as a mixture of two diastereoisomers, epimeric at the phosphate centre (the S-epimer and the R-epimer).

It is an aim of this invention to provide alternative methods of preparation of NUC-3373. It is an aim of this invention to provide methods of preparing of NUC-3373 that is stable, e.g. to long term storage in solutions. The NUC-3373 may be provided in substantially diastereomerically pure form.

Certain embodiments of this invention satisfy some or all of the above aims.

### BRIEF SUMMARY OF THE DISCLOSURE

In accordance with a first aspect of the present invention there is provided a process for the preparation of NUC-3373 (I):

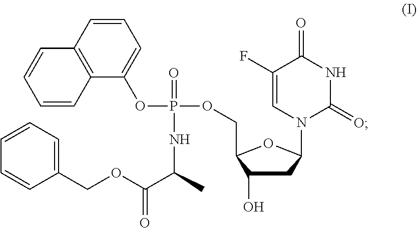

the process comprising the steps of:
a) crystallising the compound of formula (Ic) from one or more solvents

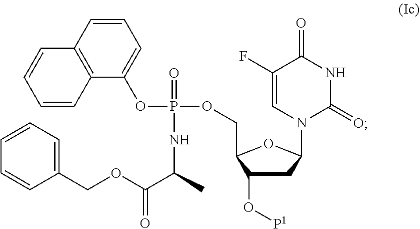

wherein P¹ is a protecting group
b) removing the protecting group P¹ from the compound of formula (Ic) to provide NUC-3373 (I).

The process may further comprise the step of:
reacting a compound of formula (Ia) with a compound of formula (Ib) to provide the compound of formula (Ic):

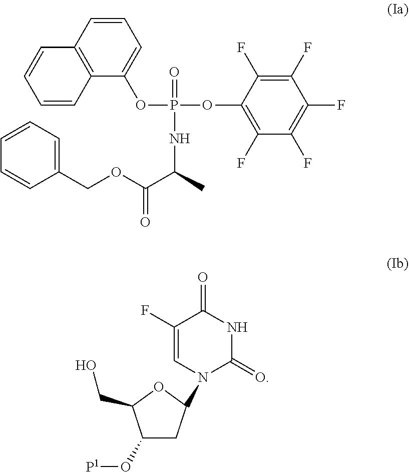

The crystallisation of the protected species (Ic), followed by deprotection results in the formation of NUC-3373 with increased stability relative to the same method in which the compound of formula (Ic) has not been crystallised before deprotection. It can offer NUC-3373 with increased stability relative, for example, to NUC-3373 obtained from a process in which crystallisation is not performed on the protected species (Ic) but in which NUC-3373 itself is subjected to purification processes, including crys.

According to some embodiments, the compound of formula (Ib) is

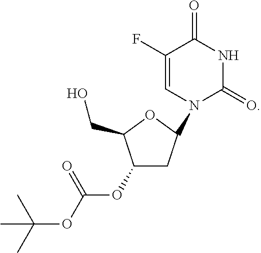

According to some embodiments, the compound of formula (Ic) is

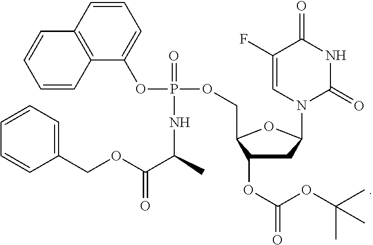

According to some embodiments, step (a) comprises the steps of:
obtaining a solution of the compound of formula (Ic) in one or more solvents;
allowing the compound of formula (Ic) to crystallise; and
recovering the crystallised compound of formula (Ic).

According to some embodiments, the step of obtaining a solution of a compound of formula (Ic) in one or more solvents comprises dissolving the compound of formula (Ic) in the one or more solvents.

According to some embodiments, the one or more solvents comprise a solvent selected from: ethanol, acetonitrile, N,N-dimethylformamide (DMF), methanol, dichloromethane (DCM), acetone, diethyl ether, toluene, n-hexane, tetrahydrofuran (THF), isopropyl alcohol (IPA), ethyl acetate, dimethylsulfoxide (DMSO), n-heptane, cyclohexane, and methyl-tert-butyl-ether (MTBE).

According to some embodiments, the one or more solvents comprises an ether e.g. diethyl ether, THF, MTBE. According to some embodiments, the ether comprises MTBE.

According to some embodiments, the one or more solvents comprises an alcohol e.g. selected from ethanol, methanol and IPA. According to some embodiments, the alcohol is IPA.

According to some embodiments, the step of allowing the compound of formula (Ic) to crystallise is conducted in the presence of an anti-solvent.

According to some embodiments, step (a) may further comprise combining an anti-solvent with the solution of a compound of formula (Ic) in one or more solvents. Step (a) may comprise adding an anti-solvent to the solution of a compound of formula (Ic) in one or more solvents. Alternatively, step (a) may comprise adding the solution of a compound of formula (Ic) in one or more solvents to an anti-solvent.

According to some embodiments, the anti-solvent is water. This might particularly be the case where the solvent is an alcohol, e.g. IPA.

According to other embodiments, the anti-solvent is an alkane, e.g. hexane, cyclohexane, pentane, petroleum ether, heptane. The anti-solvent may be heptane, e.g. n-heptane. The anti-solvent may be hexane, e.g. n-hexane. This might particularly be the case where the solvent is an ether, e.g. MTBE.

The combination of alkane (e.g. hexane or heptane) and the solution of a compound of formula (Ic) in one or more ether solvents (e.g. MTBE) will result in a mixture of ether (e.g. MTBE) and alkane (e.g. hexane) comprising the compound of formula (Ic). According to some embodiments, the mixture comprises a mixture of ether (e.g. MTBE):alkane (e.g. hexane or heptane) in a ratio from 3:1 to 1:3. According to some embodiments, the mixture comprises a mixture of ether (e.g. MTBE):alkane (e.g. hexane or heptane) in a ratio from 1:1 to 1:2.

The combination of water and the solution of a compound of formula (Ic) in one or more alcohol solvents (e.g. IPA) will result in a mixture of alcohol (e.g. IPA) and water comprising the compound of formula (Ic). According to some embodiments, the mixture comprises a mixture of alcohol (e.g. IPA):water in a ratio from 1:1 to 1:4. According to some embodiments, the mixture comprises a mixture of alcohol (e.g. IPA):water in a ratio from 1:1 to 1:3.

According to some embodiments, the step of allowing the compound of formula (Ic) to crystallise is performed at a temperature in the range of 10 to 45° C. According to some embodiments, the step of crystallising is performed at a temperature in the range of 25 to 35° C. According to some embodiments, the step of crystallising is performed at a temperature in the range of 10 to 20° C.

According to some embodiments, the step of allowing the compound of formula (Ic) to crystallise may comprise cooling the solution of the compound of formula (Ic). According to some embodiments, the step of allowing the compound of formula (Ic) to crystallise may comprise introducing a seed material to the solution.

According to some embodiments, the step of recovering the crystallised compound of formula (Ic) comprises filtration, vacuum filtration, centrifugation, solvent evaporation or crystal fishing. According to some embodiments, the step of recovering the crystallised compound of formula (Ic) comprises filtration.

According to some embodiments, step (a) may further comprise the step of drying the recovered crystallised compound of formula (Ic).

According to some embodiments, step (a) comprises the steps of:
obtaining a solution of the compound of formula (Ic) in an alcohol (e.g. IPA);
adding water to the solution of the compound of formula (Ic) in the alcohol;
allowing the compound of formula (Ic) to crystallise; and
filtering the mixture to obtain the crystallised compound of formula (Ic).

According to some embodiments, step (a) comprises the steps of:
obtaining a solution of the compound of formula (Ic) in an ether (e.g. MTBE);
adding the solution of the compound of formula (Ic) in the ether to an alkane (e.g. hexane or heptane);
allowing the compound of formula (Ic) to crystallise; and
filtering the mixture to obtain the crystallised compound of formula (Ic).

According to some embodiments, step (a) comprises the steps of:
obtaining a solution of the compound of formula (Ic) in an ether (e.g. MTBE);
adding an alkane (e.g. hexane or heptane) to the solution of the compound of formula (Ic) in the ether;
allowing the compound of formula (Ic) to crystallise; and
filtering the mixture to obtain the crystallised compound of formula (Ic).

According to some embodiments, the alkane in step (a) is heptane, e.g. n-heptane.

According to some embodiments, the step of allowing the compound of formula (Ic) to crystallise is performed for up to 4 hours. According to some embodiments, the step of allowing the compound of formula (Ic) to crystallise is performed for up to 1 hour.

The crystallisation process may be performed once. Alternatively, the crystallisation may be performed more than once, e.g. from 2 to 8 times. It maybe that more than one different set of crystallisation conditions are used.

Thus, it may be that the sequence of steps:
obtaining a solution of the compound of formula (Ic) in an alcohol (e.g. IPA);
adding water to the solution of the compound of formula (Ic) in the alcohol;
allowing the compound of formula (Ic) to crystallise; and
filtering the mixture to obtain the crystallised compound of formula (Ic),
   is repeated 2 to 4 times. In this case the crystallised compound of formula (Ic) obtained in the fourth step is dissolved in the alcohol (e.g. IPA) to obtain the solution of formula (Ic) used in the first step of the next repeat of the sequence of steps.

Likewise, it may be that the sequence of steps:
obtaining a solution of the compound of formula (Ic) in an ether (e.g. MTBE);
adding the solution of the compound of formula (Ic) in the ether to an alkane (e.g. hexane or heptane);
allowing the compound of formula (Ic) to crystallise; and
filtering the mixture to obtain the crystallised compound of formula (Ic),
   is repeated 2 to 4 times. In this case the crystallised compound of formula (Ic) obtained in the fourth step is dissolved in the ether (e.g. MTBE) to obtain the solution of formula (Ic) used in the first step of the next repeat of the sequence of steps.

It may be that the sequence of steps:
obtaining a solution of the compound of formula (Ic) in an ether (e.g. MTBE);
adding an alkane (e.g. hexane or heptane) to the solution of the compound of formula (Ic) in the ether;
allowing the compound of formula (Ic) to crystallise; and
filtering the mixture to obtain the crystallised compound of formula (Ic),
   is repeated 2 to 4 times. In this case the crystallised compound of formula (Ic) obtained in the fourth step is dissolved in the ether (e.g. MTBA) to obtain the solution of formula (Ic) used in the first step of the next repeat of the sequence of steps.

Alternatively, it may be that the at least two different crystallisation processes are performed. It may be that at least one of the crystallisation processes is one of those described above in which alcohol (e.g. IPA) is the solvent (e.g. with water as an antisolvent) and at least one of the crystallisation processes is one of those described above in which an ether (e.g. MTBE) is the solvent (e.g. with an alkane (e.g. hexane or heptane) as an antisolvent).

It may be that step (a) comprises both the steps of:
obtaining a solution of the compound of formula (Ic) in an ether (e.g. MTBE);
adding the solution of the compound of formula (Ic) in the ether to an alkane (e.g. hexane or heptane);
allowing the compound of formula (Ic) to crystallise;
filtering the mixture to obtain the crystallised compound of formula (Ic);
   and the steps of:
obtaining a solution of the compound of formula (Ic) in an ether (e.g. MTBE);
adding an alkane (e.g. hexane or heptane) to the solution of the compound of formula (Ic) in the ether;
allowing the compound of formula (Ic) to crystallise; and
filtering the mixture to obtain the crystallised compound of formula (Ic).

The two crystallisation processes may proceed in any order.

According to some embodiments, the step of reacting the compound (Ia) with the compound of formula (Ib) to provide the compound of formula (Ic), is performed in the presence of a base. The base might be a nitrogen base. Nitrogen bases include N-alkylimidazoles, (e.g. N-methyl imidazole (NMI)), imidazole, optionally substituted pyridines, (e.g. collidine, pyridine, 2,6-lutidine) and trialkylamines (e.g. triethylamine, diisopropylethylamine). Alternatively, the base may be an organometallic base or metal hydride base (e.g. NaH). Thus, the base may be a Grignard reagent (i.e. an alkylmagnesium halide). Exemplary Grignard reagents include t-butylmagnesium halides such as tBuMgCl, tBuMgBr. Preferably, the base is tBuMgCl.

In some embodiments, the step of reacting the compound (Ia) with the compound of formula (Ib) to provide the compound of formula (Ic), is performed in the presence of a solvent. The solvent may be an organic solvent. Organic solvents include but are not limited to ethers (e.g. tetrahydrofuran, dioxane, diethyl ether, methyl-t-butylether); ketones (e.g. acetone, methyl isobutyl ketone); halogenated solvents (e.g. dichloromethane, chloroform, 1,2-dichloroethane); and amides (e.g. DMF, NMP); or mixtures thereof. Where the step of reacting the compound (Ia) with the compound of formula (Ib) to provide the compound of formula (Ic) is conducted in the presence of a Grignard reagent, the organic solvent is preferably an ether. Most preferably, the solvent is tetrahydrofuran.

Where the step of reacting the compound (Ia) with the compound of formula (Ib) to provide the compound of formula (Ic) is conducted in the present of a nitrogen base, the organic solvent is most preferably a halogenated solvent or an amide.

The the step of reacting the compound (Ia) with the compound of formula (Ib) to provide the compound of formula (Ic) is typically conducted at a suitable temperature, e.g. in the range from about −5° C. to about 40° C. Preferably, the reaction temperature is in the range from about 25° C. to about 30° C. The the step of reacting the compound (Ia) with the compound of formula (Ib) may be conducted at a temperature in the range from about −5° C. to about 10° C. The reaction may be allowed to stir for a period of time from about 15 mins to about 16 h and preferably from about 30 mins to about 60 mins. The reaction may be allowed to stir for a period of time from about 15 mins to about 16 h and preferably from about 1 h mins to about 6 h.

A protecting group for a hydroxyl group (e.g. P¹) may be independently selected from optionally substituted —Si(C₁-C₆-alkyl)₃, optionally substituted —C(O)—C₁-C₆-alkyl, optionally substituted —C(O)-aryl, optionally substituted —C(O)—OC₁-C₆-alkyl, —C(O)—O-allyl, —C(O)—O—CH₂-fluorenyl, optionally substituted —C(aryl)₃, optionally substituted —(C₁-C₃-alkylene)-aryl, optionally substituted —C(O)OCH₂-aryl and —C₁-C₄-alkyl-O—C₁-C₄-alkyl.

Where a protecting group is acid sensitive (e.g. trityl, C(O)OtBu, MOM, MEM, 2,4-dimethoxybenzyl, 2,3-dimethoxybenzyl, —C(Me)₂-) the deprotection step, step b), can be conducted using a suitable acid. The acid may be a Bronsted acid (e.g. TFA, phosphoric acid, HCl, or formic acid) or a Lewis acid (e.g. ZnBr₂, CeCl₃). Lewis acids (e.g. ZnBr₂) are less preferred. HCl is likewise less preferred. Preferably, the acid is TFA.

Alternatively where the protecting group is C(O)OtBu, step b) may be achieved using a C₁-C₄-alcohol and/or water (e.g. a mixture of isopropyl alcohol (IPA) and water).

Where a protecting group is base sensitive, e.g. acetyl and benzoyl, the deprotection step can be conducted using a suitable base, e.g. aqueous NH₃ or aqueous NaOH. Base sensitive groups may however be less preferred.

Where a protecting group is a silyl group (e.g. triethylsilyl or t-butyldimethylsilyl), the deprotection step can be conducted using a suitable acid (e.g. TFA) or using a suitable fluorine source (e.g. tetrabutylammonium fluoride, fluorosilicic acid or HF).

Where a protecting group is a benzyl group or a C(O)Obenzyl group, the deprotection step can be conducted using H2 and a suitable catalyst (e.g. Pd/C). Such protecting groups may however be less preferred.

Where a protecting group is a 4-methoxy-benzyl, 2,3-dimethoxybenzyl, 2,4-dimethoxybenzyl or C(O)O-(4-methoxybenzyl) the deprotection step can be performed using a suitable oxidizing agent (e.g. meta-chloroperbenzoic acid).

Where a protecting group is —C(O)—O-allyl, the deprotection step can be performed using (PPh₃)₄Pd.

Where a protecting group is —C(O)—O—CH₂-fluorenyl, the deprotection step can be performed using piperidine.

The deprotection step may be conducted in an organic solvent or a mixture thereof. Exemplary organic solvents include, but are not limited to halogenated solvents (e.g. dichloromethane, chloroform, dichloroethane); alcohols (e.g. methanol, ethanol, isopropanol) and ethers (e.g. tetrahydrofuran, diethyl ether).

Where the deprotection step is carried out in the presence of an acid (e.g. TFA) the organic solvent is preferably a halogenated solvent, e.g. dichloromethane.

The deprotection reaction may be carried out at a temperature in the range of, for example −10° C. to about 30° C., e.g. to about 10° C. The temperature may be in the range of −5° C. to 5° C. The reaction may be allowed to stir for a period of time from about 15 mins to about 16 hours and preferably from about 1 hour to about 4 hours, and more preferably from about 2 hours to about 3 hours.

Where the deprotection step is achieved using a C₁-C₄-alcohol and/or water (e.g. a mixture of isopropyl alcohol (IPA) and water), the reaction mixture may be heated, e.g. to a temperature in the range of 30° C. to 100° C. or to a temperature in the range of 70° C. to 90° C.

Where the deprotection step is performed in the presence of an acid (e.g. TFA), isolation of the product obtained after the deprotection is typically done by quenching the excess acid used in deprotection step and extracting the product with a water immiscible organic solvent and recovering the product by evaporation of the organic solvent.

Examples of water immiscible organic solvents useful in extraction include esters such as ethyl acetate, methyl acetate, isopropyl acetate and the like; chlorinated solvents such as dichloromethane, chloroform and the like; aromatic hydrocarbon solvents such as toluene, xylene and the like; preferably ethyl acetate.

In certain embodiments, it may still be desirable to purify the compound of formula I obtained from the process of the first aspect of the invention. Methods of purification are well known to those skilled in the art and include chromatography and recrystallisation. In other embodiments, no purification is necessary.

The term ‘stable’ as used in this specification may refer to chemical stability of the compound of formula (I), e.g. when in solution or as a dry solid. It may also refer to the physical stability of solutions of the compound of formula (I). In certain embodiments, the compound of formula (I) prepared according to the process of the first aspect of the invention is stable in solution at a temperature in the range of 15 to 30° C. for up to 2 weeks, 6 months, 12 months, or longer. In certain embodiments, the compound of formula (I) is stable in solution at a temperature in the range of 2 to 8° C. for up to 2 weeks, 6 months, 12 months, or longer. Said solution may comprise DMA. In certain embodiments, the compound of formula (I) prepared according to the process of the first aspect of the invention is stable (as a dry solid) at a temperature in the range of 15 to 30° C. for up to 1 month, 6 months, 12 months, or longer. In certain embodiments, the compound of formula (I) is stable (as a dry solid) at a temperature in the range of 2 to 8° C. for up to 1 month, 6 months, 12 months, or longer. In certain embodiments, the compound of formula (I) is stable (as a dry solid) at a temperature in the range from 15 to 60° C. for up to 1 month, 6 months, 12 months, or longer. It may be that the compound of formula (I) is stable (as a dry solid) at 40° C. for at least 6 months.

In a second aspect of the invention is provided NUC-3373 obtainable (e.g. obtained) by the process of the first aspect of the invention.

In a third aspect of the invention is provided a pharmaceutical formulation comprising NUC-3373 obtainable (e.g. obtained) by the process of the first aspect of the invention. The formulation may comprise dimethyl acetamide (DMA). The formulation may also comprise water, e.g., saline.

The NUC-3373 made according to the methods of the invention may be a mixture of phosphate diastereoisomers. Alternatively, it may be the S-phosphate diastereoisomer in greater than 85% (e.g. greater than 95% or greater than 99%) diastereoisomeric purity. Alternatively, it may be the R-phosphate diastereoisomer in greater than 85% (e.g. greater than 95% or greater than 99%) diastereoisomeric purity.

The compound of formula (Ic) may be a mixture of phosphate diastereoisomers. Alternatively, it may be the S-phosphate diastereoisomer in greater than 85% (e.g. greater than 95% or greater than 99%) diastereoisomeric purity. Alternatively, it may be the R-phosphate diastereoisomer in greater than 85% (e.g. greater than 95% or greater than 99%) diastereoisomeric purity.

The compound of formula (Ia) may be a mixture of phosphate diastereoisomers. Alternatively, it may be the S-phosphate diastereoisomer in greater than 85% (e.g. greater than 95% or greater than 99%) diastereoisomeric purity. Alternatively, it may be the R-phosphate diastereoisomer in greater than 85% (e.g. greater than 95% or greater than 99%) diastereoisomeric purity.

Where the compound of formula (Ia) is a mixture of phosphate diastereoisomers, it will provide a compound of formula (Ic) (and thus NUC-3373) that is likewise a mixture of phosphate diastereoisomers. Where the compound of formula (Ia) is a single diastereoisomer, it will provide a compound of formula (Ic) (and thus NUC-3373) that is likewise a single diastereoisomer, with the reaction proceeding stereoselectively through inversion of the phosphate stereocentre.

In a fourth aspect of the invention is provided a process for the preparation of a pharmaceutical formulation of NUC-3373 (I), the process comprising:
carrying out the process of the first aspect to provide NUC-3373; and
preparing a pharmaceutical formulation comprising the NUC-3373.

The formulation may comprise a polar aprotic solvent. The formulation may comprise dimethyl acetamide (DMA). The formulation may also comprise water, e.g., saline. The step of preparing the pharmaceutical formulation may comprise dissolving the NUC-3373 in a solvent. The solvent may comprise a polar aprotic solvent. The solvent may comprise DMA. The solvent may comprise a mixture of DMA and water, e.g. saline. Where the NUC-3373 is initially dissolved in DMA, water (e.g. saline) may be added to form the formulation.

In a fifth aspect of the invention is provided a pharmaceutical formulation obtainable (e.g. obtained) by the process of the fourth aspect of the invention.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context requires otherwise. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Definitions

The term “alkyl” refers to a monovalent linear or branched saturated hydrocarbon chain. The term “C₁-C₆-alkyl” refers to an alkyl group containing from 1 to 6 carbon atoms in the hydrocarbon chain. For example, C₁-C₆-alkyl covers methyl, ethyl, n-propyl, isopropyl, n-butyl, isobutyl, n-pentyl, isopentyl, neopentyl, n-hexyl, isohexyl, 3-methylpentyl, 2,3-dimethylbutyl and neohexyl.

The term “aryl” refers to any monovalent aromatic carbocyclic ring system (i.e. a ring system containing 2(2n+1)π electrons). The aryl may be monocyclic or polycyclic. The aryl group may have from 6 to 12 carbon atoms in the ring system. Aryl groups will typically be phenyl groups. Aryl groups may however be naphthyl groups or biphenyl groups.

The group optionally substituted —Si(C₁-C₆-alkyl)₃ may be a —Si(C₁-C₄-alkyl)₃ group. The group is (i.e. the alkyl groups are) preferably unsubstituted. Illustrative examples include triethylsilyl and t-butyl-dimethylsilyl.

The group optionally substituted —C(O)—C₁-C₆-alkyl may be a —C(O)—C₁-C₄-alkyl group. The group (i.e. the alkyl group) is preferably unsubstituted. Illustrative examples include acetyl and propionyl.

The group optionally substituted —C(O)-aryl may be a —C(O)-phenyl group. The group (i.e. the phenyl group) is preferably unsubstituted. Illustrative examples include benzoyl.

The group optionally substituted —C(O)—OC₁-C₆-alkyl may be a —C(O)—OC₁-C₄-alkyl group. The group (i.e. the alkyl group) is preferably unsubstituted. Illustrative examples include —C(O)—O-methyl and —C(O)—O-ethyl. A particularly preferred example is C(O)OtBu.

The group optionally substituted —(C₁-C₃-alkylene)-aryl is preferably an optionally substituted benzyl group. Illustrative examples include benzyl, phenethyl, 4-methoxy benzyl, 4-nitrobenzyl, 4-bromobenzyl, 2,3-dimethoxybenzyl and 2,4-dimethoxybenzyl.

The group optionally substituted —C(O)OCH₂-aryl is preferably an optionally substituted —C(O)Obenzyl group. Illustrative examples include —C(O)Obenzyl and —C(O)O-(4-methoxybenzyl).

The group optionally substituted —C₁-C₄-alkyl-O—C₁-C₄-alkyl may be a —C₁-C₂-alkyl-O—C₁-C₂-alkyl group. The group is (i.e. the alkyl groups are) preferably unsubstituted. Illustrative examples include methoxy-methyl (MOM) and 2-methoxy-ethoxy-methyl (MEM).

The group optionally substituted —S(O)₂—C₁-C₆-alkyl may be a —S(O)₂—C₁-C₄-alkyl group. The group (i.e. the alkyl group) is preferably unsubstituted. Illustrative examples include methanesulfonate.

The group optionally substituted —S(O)₂-aryl may be a —S(O)₂-phenyl group. Illustrative examples include phenylsulfonate, 4-methylphenylsulfonate and 4-nitro phenylsulfonate.

The group optionally substituted —C(aryl)₃ may be a —C(phenyl)₃ group. Illustrative examples include trityl.

Throughout this specification, ‘diastereomerically enriched form’ and ‘substantially diastereomerically pure form’ means a diastereoisomeric purity of greater than 95%. ‘Diastereomerically enriched form’ and ‘substantially diastereomerically pure form’ may mean a diastereoisomeric purity of greater than 98%, greater than 99% or greater than 99.5%.

Any of the aforementioned alkyl groups, are optionally substituted, where chemically possible, by 1 to 3 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a} C(O)R^{a} and CONR^{a}R^{a}; wherein R^{a} is independently at each occurrence selected from H, C₁-C₄ alkyl and C₁-C₄ haloalkyl.

It may be that any of the aforementioned alkyl groups, are optionally substituted, where chemically possible, by 1 to 3 substituents which are each independently at each occurrence selected from the group consisting of: halo, nitro, cyano, NR^{a}R^{a}, OR^{a} and SR^{a}, wherein R^{a} is independently at each occurrence selected from H, C₁-C₄ alkyl and C₁-C₄ haloalkyl.

It may be that any of the aforementioned alkyl groups is unsubstituted.

Any of the aforementioned aryl (e.g. phenyl, including the phenyl groups in benzyl groups) groups, are optionally substituted, where chemically possible, by 1 to 3 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a} C(O)R^{a}, CONR^{a}R^{a}, C₁-C₄-alkyl, C₂-C₄-alkenyl, C₂-C₄-alkenyl, and C₁-C₄ haloalkyl; wherein R^{a} is independently at each occurrence selected from H, C₁-C₄ alkyl and C₁-C₄ haloalkyl.

It may be that any of the aforementioned aryl groups (e.g. phenyl, including the phenyl groups in benzyl groups) are optionally substituted, where chemically possible, by 1 to 3 substituents which are each independently at each occurrence selected from the group consisting of: halo, nitro, cyano, NR^{a}R^{a}, NR^{a}S(O)₂R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a} C(O)R^{a}, CONR^{a}R^{a}, C₁-C₄-alkyl, C₂-C₄-alkenyl, C₂-C₄-alkenyl, and C₁-C₄ haloalkyl; wherein R^{a} is independently at each occurrence selected from H, C₁-C₄ alkyl and C₁-C₄ haloalkyl.

It may be that any of the aforementioned aryl (e.g. phenyl, including the phenyl groups in benzyl groups) groups are optionally substituted by 1 to 3 substituents which are each independently at each occurrence selected from the group consisting of: halo, nitro, OR^{a}; C₁-C₄-alkyl, C₁-C₄ haloalkyl; wherein R^{a} is independently at each occurrence selected from H, C₁-C₄ alkyl and C₁-C₄ haloalkyl.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the specification appended hereto.

The following abbreviations are used throughout this specification:

| | |
|---|---|
| ACN - acetonitrile | Boc - t-butylcarbonate |
| DCM - dichloromethane | DMSO - dimethylsulfoxide |
| DM (water) - demineralised | DMA - dimethylacetamide |
| DMF - N,N-dimethylformamide | FUDR - 5-fluoro-2′-deoxyuridine |
| HDPE - high-density polyethylene | IPA - isopropyl alcohol |
| LDPE - low-density polyethylene | MEM - 2-methoxyethoxymethyl |
| MOM - methoxymethyl | MTBE - methyl-t-butylether |
| NMI - N-methyl imidazole | NMP - N-methyl-2-pyrrolidone |
| RRT - relative retention time | RT - room temperature |
| TBAF - tetrabutylammonium fluoride | TBDMS - tert-butyldimethylsilyl |
| TFA - trifluoroacetic acid | THF - tetrahydrofuran |
| TsOH - | |
| para-toluene sulfonic | |
| (tosic) acid | |
| HPLC - | |
| high-performance | |
| liquid chromatography | |
| UPLC - | |
| ultra high performance | |
| liquid chromatography | |

### EXAMPLES

The present invention is further illustrated by the following examples, which are provided by way of illustration only and should not be construed to limit the scope of the invention.

### Example 1: Preparation of a Compound of Formula Ic

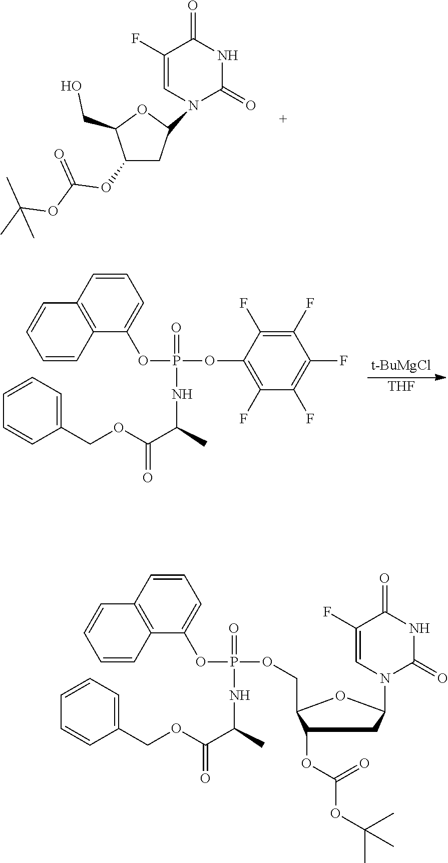

To a mixture of 3′-Boc-floxuridine 1 (90 g; WO2019/053476; WO2018/229493) in THE (720 mL) was added PFP ligand (mixture of phosphate diastereoisomers; 150.48 g; WO2018/229493) at 30° C. A further 135 mL of THE was added at 30° C. and the reaction was cooled to 0° C. 299 mL of 2.0 M tert-Butyl magnesium chloride in THE was added to the reaction at 0° C. over approximately 60 minutes. A further 45 mL of THE was added at 0° C. The reaction was maintained for 3-4 h at 0° C.

A second reaction vessel was charged with 846 mL of water and 54 mL of hydrochloric acid and cooled to 5° C. The contents of the first reaction vessel were added to the second reaction vessel at 5° C. over approximately 60 minutes. 180 mL of THE was added to the second reaction vessel and maintained for 2-3 h with the temperature adjusted to 5° C. 1350 mL of ethyl acetate was added to the second reaction vessel at 5° C., then the temperature of the second reaction vessel was raised to 30° C. The reaction mass was allowed to settle into an aqueous layer (bottom) and an organic layer (top) over 30 minutes. The layers were then separated with extraction using water. The organic layer was added to a sodium chloride solution at the same temperature. The solvent was removed under vacuum at below 45° C. To the resulting brown syrup was added 1530 mL of MTBE at 30° C. and maintained for 20-30 minutes before filtering through celite and concentrating in vacuo.

### Example 2: Crystallisation of a Compound of Formula Ic

1800 mL of MTBE was added to the product of Example 1 at 25° C. The resultant solution was added slowly to 2700 mL of n-heptane. The reaction mass was kept for 3-5 h at 25° C. The mixture was filtered to obtain a solid which was dried under vacuum. 1800 mL of MTBE was added to the solid at 25° C. and 2700 mL of n-heptane was added slowly to the resultant solution. The reaction mass was kept for 3-5 h at 25° C. The mixture was filtered to obtain a solid which was dried under vacuum. The solid was dissolved in 900 mL of isopropyl alcohol and the solution held at 40° C. for 20-30 minutes before being cooled to 25° C. 1800 mL water was added and the reaction was held at 25° C. for 2-3 h before being filtered. The solids were again dissolved in 900 mL of isopropyl alcohol and the solution held at 40° C. for 20-30 minutes before being cooled to 25° C. 1800 mL water was added and the reaction was held at 25° C. for 2-3 h before being filtered. The solid was dried under vacuum.

Yield: 143.3 g. Purity by HPLC (% area): 99.45%.

### Example 3: Preparation of NUC-3373 (I)

130 g of 3′-Boc NUC-3373 (the product of Example 2) was dissolved in 975 mL of Isopropyl alcohol and 1950 mL of water and the mixture was heated to 80° C. and held at 80° C. for 7-9 h. The mixture was cooled to 30° C. and 975 mL of isopropyl alcohol and 1300 mL of cyclohexane were added. The aqueous layer and the organic layer were separated. The aqueous layer was mixed with 1300 mL of cyclohexane and the aqueous layer and the organic layer were separated. To the aqueous layer was added sodium bicarbonate solution [prepared by dissolving 65.0 g of sodium bicarbonate in 950 mL of DM water at 30° C.] and 1300 mL of ethyl acetate. The aqueous layer was again mixed with 1300 mL of ethyl acetate and the layers separated. The combined organic layers from the two ethyl acetate separations were washed with NaCl and concentrated in vacuo to give NUC-3373.

Yield: 78.0 g

### Comparative Example 4, Part 1: Preparation of a Compound of Formula Ic

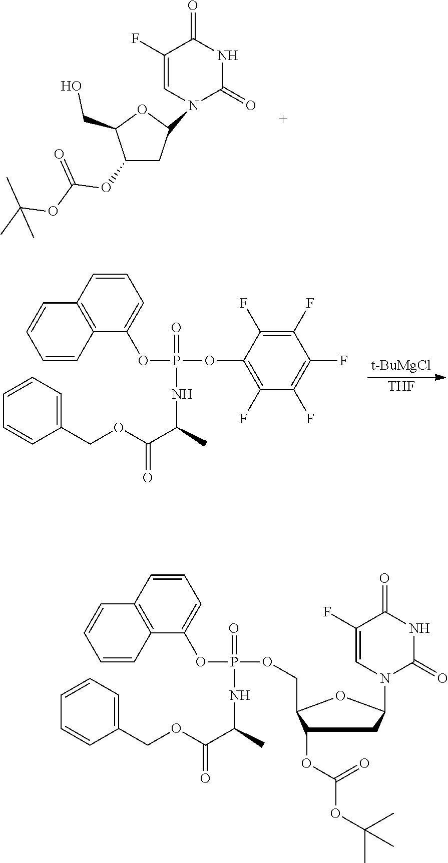

To a mixture of 3′-Boc-floxuridine 1 (10 g) in THE (80 mL) was added PFP ligand (mixture of phosphate diastereoisomers; 19.11 g) at 30° C. A further 20 mL of THF was added at 30° C. and the reaction was cooled to 5° C. 36.10 mL of 2.0 M tert-Butyl magnesium chloride in THF was added to the reaction at below 15° C. The reaction was stirred for 3-4 h at 20° C.

100 mL of 10% ammonium chloride solution was added dropwise and the reaction was stirred for 15 minutes at 30° C. 100 mL of ethyl acetate was added to the reaction vessel. The reaction mass was allowed to settle into an aqueous layer (bottom) and an organic layer (top) over 30 minutes. The layers were then separated with extraction using water. The organic layer was added to a sodium chloride solution at the same temperature. The reaction mass was allowed to settle into an aqueous layer (bottom) and an organic layer (top) over 30 minutes. The layers were then separated and the solvent was removed from the organic layer under vacuum at below 45° C.

### Comparative Example 4, Part 2: Preparation of NUC-3373 (I)

The product of Comparative Example 4, part 1 (3′-Boc NUC-3373) was dissolved in 150 mL of DCM. 50 mL of trifluoroacetic acid was added dropwise and the mixture was heated to 30° C. and held at 30° C. for 3-4 h. The mixture was cooled to 5° C. and 150 mL of DM water was added. The aqueous layer and the organic layer were separated. The aqueous layer was mixed with 100 mL of DCM and the organic layer was separated. To the combined organic layers was added sodium bicarbonate solution [prepared by dissolving 7 g of sodium bicarbonate in 100 mL of DM Water at 30° C.]. The aqueous layer and the organic layer were separated. The organic layer was washed with NaCl, followed by a wash with sodium sulphate before being concentrated in vacuo to give NUC-3373.

The product was purified by column chromatography, with Silica as the stationary phase and ethyl acetate and DCM as the mobile phase. Fractions were monitored by TLC. Pure fractions were combined and the solvent was removed under vacuum at below 45° C.

15 mL of ethyl acetate was added to the product at 30° C. The resultant solution was added dropwise to 180 mL of cyclohexane at 7° C. The reaction mass was stirred for 3-4 h at 0° C. The mixture was filtered to obtain a solid which was dried under vacuum. 100 mL of DCM was added to the solid at 30° C. 1 g of activate carbon was added to the resultant solution which was stirred for 30-45 mins before being filtered. The solvent was then removed under vacuum at below 45° C. 50 mL of DCM was added to the solid at 30° C. The solvent was then removed under vacuum at below 45° C. 50 mL of cyclohexane was added to the solid at 30° C. The solvent was then removed and the solid was dried under vacuum at below 45° C.

Yield: 9.7 g

### Example 5: Stability Data

### 5.1: 12 Month Dry Powder

NUC-3373 prepared according to Example 3, above, was packed into a transparent LDPE bag which was then filled with nitrogen and tied with a strip seal. This bag was then placed into another transparent LDPE bag which was also filled with nitrogen. A silica gel packet was placed between the two bags before the outermost bag was tied with a strip seal. These bags were then placed in a triple laminated sun light barrier bag which was sealed with a heat sealer. The bags were kept in a HDPE container. The NUC-3373 was initially assayed by HPLC after manufacture then after 3, 6, 9 and 12 months of storage. The stability data is provided in Table 1.

TABLE 1

HPLC assay (% w/w on anhydrous
and solvent free basis) at certain
storage temperatures

| Time | 5 ± 3° C. | 25 ± 2° C. |
|---|---|---|
| Initial | 99.6 | 99.6 |
| 3^{rd} month | 99.9 | 99.6 |
| 6^{th} month | 100.9 | 100.7 |
| 9^{th} month | 101.5 | 101.5 |
| 12^{th} month | 99.4 | 99.4 |

The data in Table 1 shows that NUC-3373 prepared according to the process of the present invention is chemically stable for at least 12 months at both 2-8° C. and 15-30° C.

### 5.2 6 Month Dry Powder

NUC-3373 prepared according to Example 3 and Comparative Example 4 was packed into a transparent LDPE bag which was then filled with nitrogen and tied with a strip seal. This bag was then placed into another transparent LDPE bag which was also filled with nitrogen. A silica gel packet was placed between the two bags before the outermost bag was tied with a strip seal. These bags were then placed in a triple laminated sun light barrier bag which was sealed with a heat sealer. The bags were kept in a HDPE container.

NUC-3373 was initially assayed by HPLC after manufacture (0 months) then after 1 and 2 and/or 3 and 6 months of storage at 2-8° C., 25° C. or 40° C. The stability data is provided in Table 2.

TABLE 2

| | HPLC assay (% w/w on anhydrous and solvent free basis) | | | | | | | | | | | |
|---|---|---|---|---|---|---|---|---|---|---|---|---|
| Storage | Comparative | | | Example 3, | | | Example 3, | | | Example 3, | | |
| temp. | Example 4 | | | Batch 1 | | | Batch 2 | | | Batch 3 | | |
| (° C.) | 2-8 | 25 | 40 | 2-8 | 25 | 40 | 2-8 | 25 | 40 | 2-8 | 25 | 40 |
| Storage | | | | | | | | | | | | |
| time | | | | | | | | | | | | |
| (months) | | | | | | | | | | | | |
| 0 | 100 | 99.7 | 99.7 | 100.4 | 100.4 | 100.4 | 100.2 | 100.2 | 100.2 | 100.3 | 100.3 | 100.3 |
| 1 | — | — | 99.3 | — | — | 100.0 | — | — | 99.7 | — | — | 100.6 |
| 2 | — | — | 99.0 | — | — | 101.5 | — | — | 100.9 | — | — | 101.5 |
| 3 | 100 | 99.5 | 99.3 | 101.5 | 101.1 | 101.6 | 101.3 | 101.1 | 101.5 | 101.9 | 101.8 | 102.2 |
| 6 | 100 | 99.6 | 97.7 | 100.4 | 100.8 | 100.7 | 100.4 | 100.7 | 100.4 | 100.5 | 101.0 | 100.8 |
| Decrease | None | 0.1 | 2.0 | None | None | None | None | None | None | None | None | None |
| after 6 | | | | | | | | | | | | |
| months | | | | | | | | | | | | |

The Alpha Naphthol content of the same batches was also determined by HPLC. The levels of the alpha naphthol impurity is a key indicator of stability of the tested material. Data is provided in Table 3.

TABLE 3

| | Alpha Naphthol by HPLC (% w/w) | | | | | | | | | | | |
|---|---|---|---|---|---|---|---|---|---|---|---|---|
| | Comparative | | | | | | | | | | | |
| Storage | Example 4 | | | Example 3, Batch 1 | | | Example 3, Batch 2 | | | Example 3, Batch 3 | | |
| temp. (° C.) | 2-8 | 25 | 40 | 2-8 | 25 | 40 | 2-8 | 25 | 40 | 2-8 | 25 | 40 |
| Storage | | | | | | | | | | | | |
| time | | | | | | | | | | | | |
| (months) | | | | | | | | | | | | |
| 0 | 0.09 | 0.09 | 0.09 | 0.15 | 0.15 | 0.15 | 0.44 | 0.44 | 0.44 | 0.18 | 0.18 | 0.18 |
| 1 | — | — | 0.18 | — | — | 0.15 | — | — | 0.44 | — | — | 0.17 |
| 2 | — | — | 0.27 | — | — | 0.15 | — | — | 0.42 | — | — | 0.17 |
| 3 | 0.10 | 0.10 | 0.18 | 0.16 | 0.15 | 0.14 | 0.46 | 0.45 | 0.42 | 0.18 | 0.18 | 0.17 |
| 6 | 0.09 | 0.10 | 1.2 | 0.15 | 0.14 | 0.13 | 0.43 | 0.42 | 0.38 | 0.17 | 0.17 | 0.15 |
| Increase | None | 0.1 | 1.11 | None | None | None | None | None | None | None | None | None |
| after 6 | | | | | | | | | | | | |
| months | | | | | | | | | | | | |

The data in Tables 2 and 3 show that NUC-3373 prepared according to the process of the present invention (i.e. where protected NUC-3373 is purified by crystallisation prior to deprotection) is thermally more stable than those prepared by other methods, including those in which NUC-3373 itself is subjected to extensive purification once it has been deprotected but not beforehand.

### 5.3 Solution Stability

Stability data is also provided in Table 4, which compares the levels of alpha naphthol (0.60 RRT) present in solutions of 400 mg/mL NUC-3373, prepared by Example 3 or 4, above, in DMA:Saline (80:20 v/v) at different temperatures measured over set periods of time. The tested solutions are suitable for infusion and injection and the temperatures and times measured are representative of the environments that solutions suitable for infusion and injection would typically be stored in.

In Table 4 the two values provided for each temperature at each time period, separated by “/”, represent different preparations (samples) of that synthesis.

TABLE 4
0.60 RRT (Alpha Naphthol)

| | Comparative | Example 3 | Example 3 |
|---|---|---|---|
| Time | Example 4 | Batch 1 | Batch 2 |
| Initial | 0.02 | 0.07 | 0.01 |
| After 15 days at 2-8° C. | 0.04/0.04 | 0.07/0.07 | 0.01/0.01 |
| After 1 month at 2-8° C. | 0.04/0.04 | 0.07/0.07 | 0.02/0.02 |
| After 3 months at 2-8° C. | 0.06/0.06 | 0.07/0.07 | 0.02/0.02 |
| After 6 months at 2-8° C. | — | 0.07/0.08 | 0.03/0.02 |
| After 9 months at 2-8° C. | — | 0.07/0.08 | 0.03/0.03 |
| After 12 months at 2-8° C. | — | — | 0.04/0.04 |
| After 15 days at 25 ± 5° C. | 0.19/0.20 | 0.07/0.08 | 0.03/0.02 |
| After 1 month at 25 ± 5° C. | 0.29/0.29 | 0.08/0.08 | 0.04/0.04 |
| After 3 months at | 0.45/0.46 | 0.10/0.11 | 0.11/0.11 |
| 25 ± 5° C. | | | |
| After 6 months at | — | 0.20/0.22 | 0.34/0.35 |
| 25 ± 5° C. | | | |
| After 9 months at | — | 0.42/0.38 | 0.89/0.89 |
| 25 ± 5° C. | | | |
| After 12 months at | — | — | 1.60/1.60 |
| 25 ± 5° C. | | | |

The data in Table 4 shows that solutions comprising NUC-3373 prepared according to the process of the present invention (i.e. where the protected NUC-3373 is purified by crystallisation) are more stable than those prepared by other methods, including those in which NUC-3373 itself is subjected to extensive purification once it has been deprotected but not beforehand. This is particularly the case when the samples are stored at room temperature.

The inventors have found in further work that DMA:Saline (80:20 v/v) solutions of samples obtained according to the process of the invention show no loss of purity or assay, and alpha naphthol impurity up to 12 months at 2-8° C. This stability data (obtained by UPLC) is provided in Table 5.

TABLE 5

| Time at 2-8° C. | Assay | Purity | Total Impurities |
|---|---|---|---|
| Initial | 101% | 99.9% | 0.07% |
| 3^{rd} month | 99% | 99.9% | 0.07% |
| 6^{th} month | 101% | 99.9% | 0.08% |
| 12^{th} month | 101% | 99.9% | 0.09% |

The data in Table 5 shows that solutions comprising NUC-3373 prepared according to the process of the present invention are both chemically and physically stable for at least 12 months.

We claim:

1. A process for the preparation of NUC-3373 (I): 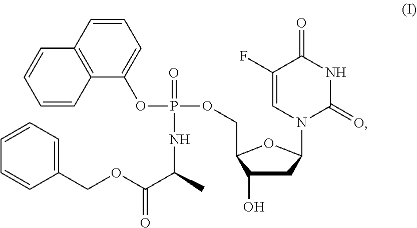
the process comprising the steps of:
a) crystallizing a compound of formula (Ic) from one or more solvents; 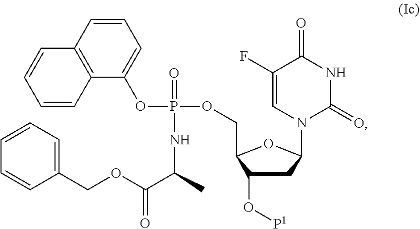
wherein P¹ is a protecting group; and
b) removing the protecting group P¹ from the compound of formula (Ic) to provide NUC-3373 (I).

2. The process of claim 1, wherein the process further comprises the step of:
reacting a compound of formula (Ia) with a compound of formula (Ib) to provide the compound of formula (Ic):

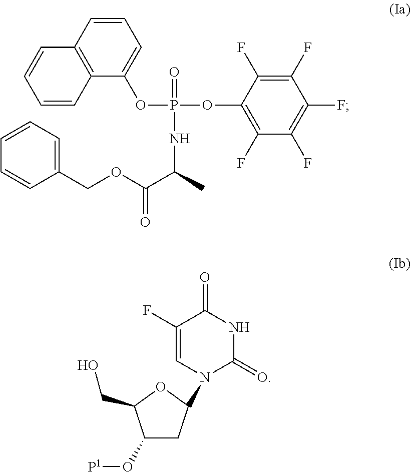

3. The process according to claim 2, wherein P¹ is C(O)O-t-Bu.

4. The process according to claim 3, wherein the one or more solvents in step a) comprise a solvent selected from the group consisting of: water, ethanol, acetonitrile, DMF, methanol, DCM, acetone, diethyl ether, toluene, n-hexane, THF, IPA, ethyl acetate, DMSO, n-heptane, cyclohexane, and MTBE or a mixture thereof.

5. The process according to claim 1, wherein step a) comprises the steps of:
(i) obtaining a solution of the compound of formula (Ic);
(ii) allowing the compound of formula (Ic) to crystallize; and
(iii) recovering the crystallized compound of formula (Ic).

6. The process according to claim 5, wherein step a) comprises the steps of:
(i) obtaining a solution of the compound of formula (Ic) in an alcohol;
(ii) adding water to the solution of the compound of formula (Ic) in an alcohol;
(iii) allowing the compound of formula (Ic) to crystallize; and
(iv) filtering the mixture to obtain the crystallized compound of formula (Ic).

7. The process according to claim 6, wherein the alcohol is IPA.

8. The process according to claim 5, wherein step a) comprises the steps of:
(i) obtaining a solution of a compound of formula (Ic) in an ether;
(ii) adding the solution of a compound of formula (Ic) in an ether to an alkane;
(iii) allowing the compound of formula (Ic) to crystallize; and
(iv) filtering the mixture to obtain the crystallized compound of formula (Ic).

9. The process according to claim 5, wherein step a) comprises the steps of:
(i) obtaining a solution of a compound of formula (Ic) in an ether;
(ii) adding an alkane to the solution of a compound of formula (Ic) in an ether;
(iii) allowing the compound of formula (Ic) to crystallize; and
(iv) filtering the mixture to obtain the crystallized compound of formula (Ic).

10. The process according to claim 9, wherein the ether is MTBE.

11. The process according to claim 10, wherein the alkane is hexane.

12. The process according to claim 10, wherein the alkane is n-heptane.

13. The process according to claim 1, wherein the process comprises at least one crystallization process as described in claim 6 and at least one crystallization process as described in claim 8.

14. A compound of structure: 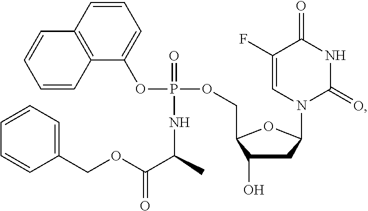
prepared by the process of claim 1.

15. A pharmaceutical formulation comprising NUC-3373 (I): 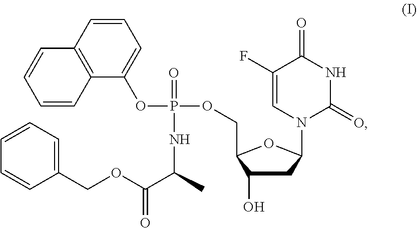
obtainable by the process of claim 1.

16. The pharmaceutical formulation of claim 15, wherein the formulation further comprises dimethyl acetamide (DMA).

17. A process for the preparation of a pharmaceutical formulation of NUC-3373 (I): 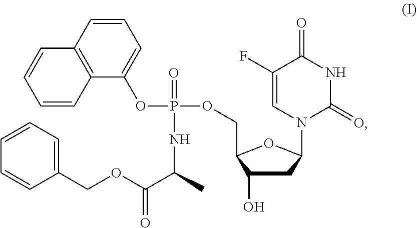
wherein the process comprises:
(i) carrying out the process of claim 1 to provide NUC-3373 (I); and
(ii) preparing a pharmaceutical formulation comprising the NUC-3373 (I).

18. The process of claim 17, wherein the pharmaceutical formulation comprises DMA.

19. A pharmaceutical formulation of NUC-3373 (I): 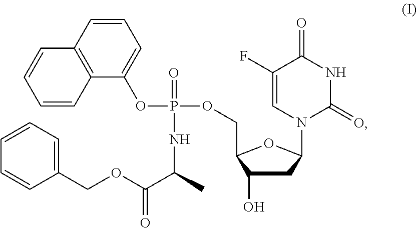
obtainable by the process of claim 17.
